(19) United States
(12) Patent Application Publication
(10) Pub. No.: US 2025/0064876 A1
(43) Pub. Date: Feb. 27, 2025

(54) NEBULIZED INHALATION LIQUID FOR RELIEVING RHINITIS AND PREPARATION METHOD THEREOF, AND NEBULIZER

(71) Applicant: Changde Jizhi Biological Technology Co., Ltd, Changde (CN)

(72) Inventor: Xinqiao HE, Changde (CN)

(21) Appl. No.: 18/452,614

(22) Filed: Aug. 21, 2023

(51) Int. Cl. A61K 36/23; A61K 33/10; A61K 33/30; A61K 33/42; A61K 36/287; A61K 36/31; A61K 36/82; A61K 36/88; A61M 11/00

(52) U.S. Cl. CPC A61K 36/23; A61K 33/10; A61K 33/30; A61K 33/42; A61K 36/287; A61K 36/31; A61K 36/82; A61K 36/88; A61M 11/005; A61K 2236/19; A61K 2236/39

(57) ABSTRACT

Provided are nebulized inhalation liquid for relieving rhinitis, a nebulizer, and a preparation method of nebulized inhalation liquid for relieving rhinitis, which belong to biomedical technology. Raw materials of the nebulized inhalation liquid include celery seed, spring onion seed, watercress, crowndaisy Chrysanthemum, water hyacinth, monazite, zinc oxide and calcium carbonate. The research shows that the nebulized inhalation liquid has a good auxiliary treatment effect on rhinitis.

### STATEMENT REGARDING PRIOR DISCLOSURES BY THE INVENTOR OR A JOINT INVENTOR

Chinese Publication No. CN115475208A dated Dec. 16, 2022 is a grace period inventor-originated disclosure which qualifies as an exception under 35 U.S.C. 102(b)(1).

### TECHNICAL FIELD

The disclosure relates to the technical field of biomedicine, particularly to nebulized inhalation liquid for relieving rhinitis and a preparation method thereof, and a nebulizer.

### BACKGROUND

Rhinitis refers to inflammation of nasal mucosa and submucosa. Rhinitis is characterized by congestion or edema, and a patient with rhinitis often has symptoms such as nasal congestion, watery nose, itchy nose, throat discomfort, and cough. Most of the symptoms include the nasal congestion, runny nose, sneezing, headache, and dizziness. One characteristic of the nasal congestion is intermittent, for example, during the day, when it is hot and the patient is working or exercising, the nasal congestion is relieved, while at night, when the patient is sitting still or it is cold, the nasal congestion is aggravated. Another characteristic of the nasal congestion is alternation, for example, when the patent is lying on the side, a lower nasal cavity is blocked and an upper nasal cavity is well ventilated. Because of the nasal congestion, occasional hyposmia, headache, dizziness, and occlusive nasal sound, sleep is affected.

It is best to treat rhinitis according to a cause therefor. A common rhinitis is allergic rhinitis, which is caused by pollen in spring or mites in the room. Methods for treating rhinitis include four methods as follows. For a first method, anti-allergic treatment is mainly performed, an antihistamine such as loratadine (C₂₂H₂₃ClN₂O₂), ebastine (C₃₂H₃₉NO₂), or cetirizine (C₂₁H₂₅ClN₂O₃) can be used, and a leukotriene receptor blocker can also be used, a common one of the leukotriene receptor blocker is montelukast sodium (C₃₅H₃₅ClNNaO₃S). If rhinitis is serious, both of the antihistamine and the leukotriene receptor blocker may be used at the same time, and if rhinitis is not serious, one of the antihistamine and the leukotriene receptor blocker may be used to control the corresponding symptoms. For a second method, it is suggested to use locally a nasal spray. The nasal spray can be divided into a hormone nasal spray and a non-hormone nasal spray. The non-hormone nasal spray commonly includes levocabastine hydrochloride (C₂₆H₃₀ClFN₂O₂) and azelastine (C₂₂H₂₄ClN₃O). The hormone nasal spray includes a budesonide nasal spray, a fluticasone propionate nasal spray and a mometasone furoate aqueous nasal spray, a good curative effect can be achieved by using oral medicine combined with the nasal spray. For a third method, it is recommended to carry out nasal irrigation treatment, through which the retention of nasal allergic substances is reduce through irrigation, secretions are flushed out, therefore, corresponding symptoms can be quickly relieved. This method can be used for allergic rhinitis, and also for chronic rhinitis. Symptoms of the chronic rhinitis are mainly repeated nasal congestion, and sometimes there will be white sticky or yellow pus, and in this case, there is generally no special medicine. For a third method, it is suggested that Chinese medicines related to the rhinitis can be taken orally, and the hormone nasal sprays can be used locally for regular treatment for a period. If an effect is not good and the situation of inferior nasal concha hypertrophy has occurred, a surgery may be further needed. However, all the above treatment methods have some side effects, or need long-term treatment, some symptoms of rhinitis cannot be relieved well.

It is of great significance to research and develop a novel product for reliving rhinitis in providing new choices for clinical application.

### SUMMARY

Based on the above reasons, the applicant has obtained novel nebulized inhalation liquid for relieving rhinitis after several creative labors. Raw materials of the nebulized inhalation liquid include celery seed, spring onion seed, watercress, crowndaisy Chrysanthemum, water hyacinth, monazite, zinc oxide (ZnQ) and calcium carbonate (CaCO₃). The research demonstrates that the nebulized inhalation liquid of the disclosure has a good auxiliary treatment effect on rhinitis. The disclosure is realized by the following technical solutions.

Nebulized inhalation liquid for relieving rhinitis is provided, and raw material of the nebulized inhalation liquid include celery seed, spring onion seed, watercress, crowndaisy Chrysanthemum, water hyacinth, monazite, zinc oxide and calcium carbonate.

In an embodiment, the celery seed is 300-500 parts by weight, the spring onion seed is 40-80 parts by weight, the watercress is 400-600 parts by weight, the crowndaisy Chrysanthemum is 350-550 parts by weight, the water hyacinth is 150-350 parts by weight, the monazite is 400-600 parts by weight, the zinc oxide is 400-600 parts by weight, and the calcium carbonate is 300-400 parts by weight.

In an embodiment, the celery seed is 400 parts by weight, the spring onion seed is 60 parts by weight, the watercress is 500 parts by weight, the crowndaisy Chrysanthemum is 450 parts by weight, the water hyacinth is 250 parts by weight, the monazite is 500 parts by weight, the zinc oxide is 500 parts by weight, and the calcium carbonate is 350 parts by weight.

In an embodiment, the nebulized inhalation liquid is applied to prepare a medicine for adjuvant treatment of rhinitis.

In an embodiment, a preparation method of the nebulized inhalation liquid includes the following steps 1 to 3:
step 1, crushing the celery seed, the spring onion seed, the watercress, the crowndaisy Chrysanthemum, and the water hyacinth into a slurry; filtering the slurry to remove a residue of the slurry and thus obtain a filtrate; heating and boiling the filtrate for 50-70 minutes to obtain a heated and boiled filtrate, and filtering the heated and boiled filtrate to obtain a filtered filtrate; adding a fructo-oligosaccharide and an isomaltooligosaccharide into the filtered filtrate, where a weight of the fructo-oligosaccharide and the isomaltooligosaccharide is 0.1-0.3 times that of the filtered filtrate, and a weight ratio of the fructo-oligosaccharide to the isomaltooligosaccharide is 1:1; performing uniformly stirring on the filtered filtrate added with the fructo-oligosaccharide and the isomaltooligosaccharide to obtain a stirred filtrate, placing the stirred filtrate in a fermentation tank for a first fermentation to obtain fermentation liquid I, where a fermentation temperature for the first fermentation is in a range of 18 degrees Celsius (° C.)−25° C., and a fermentation period for the first fermentation is in a range of 16-24 days; taking out and filtering the fermentation liquid I to obtain a filtrate II, placing the filtrate II in the fermentation tank, and adding the fructo-oligosaccharide in the fermentation tank for a second fermentation to obtain fermentation liquid II, where a weight of the fructo-oligosaccharide for the second fermentation is 0.1-0.2 times of a weight of the filtrate II, a fermentation temperature for the second fermentation is 18° C.-25° C., and a fermentation period for the second fermentation is 16-24 days; taking out and filtering the fermentation liquid II to obtain a filtrate III, placing the filtrate III in the fermentation tank, adding the isomaltooligosaccharide to the filtrate III for a third fermentation to obtain fermentation liquid III, where a weight of the added isomaltooligosaccharide for the third fermentation is 0.1-0.2 times that of the filtrate III, a fermentation temperature for the third fermentation is 18° C.-25° C., and a fermentation period for the third fermentation is 16-24 days; and distilling the fermentation liquid III to a distilling temperature of 25° C. with a relative density of 1.20-1.25, and thereby obtaining a fermented product;
step 2, pulverizing the monazite, the calcium carbonate and the zinc oxide and uniformly mixing to obtain a powder; placing the powder in a furnace at a temperature of 1800° C.-2200° C. for firing for 80-100 minutes to obtain a fired powder; and placing the fired powder in a 1000-mesh cloth bag and performing distilling with a steamer until there is no distillate, to thereby obtain a distilled liquid; and
step 3, completely mixing the fermented product and the distilled liquid to obtain a mixture; extracting the mixture by using Camellia oil to obtain a Camellia oil layer, where a weight of the Camellia oil is 2-3 times that of the fermented product and the distilled liquid; and adding glycerol (C₃H₈O₃), 1,2-propylene glycol (C₃H₈O₂) and structured water into the Camellia oil layer to obtain the nebulized inhalation liquid. The structured water is also referred to small molecular group water, micro cluster water, small cluster water, or micro clustered water; and within 100 hertz (Hz), the structured water is composed of 5-7 water molecules.

In an embodiment, the glycerol is 30-40 parts by weight, the 1,2-propylene glycol is 20-30 parts by weight, and the structured water is 30-60 parts by weight.

In an embodiment, the glycerol is 35 parts by weight, the 1,2-propylene glycol is 25 parts by weight, and the structured water is 45 parts by weight.

In an embodiment, a nebulizer is provided, which includes a micro ultrasonic atomizing head, a button cell or a rechargeable lithium battery, and a liquid storage bottle.

The nebulized inhalation liquid can be applied to a medical nebulizer in a prior art for patients with rhinitis.

The nebulized inhalation liquid can be also applied to a nebulizer similar to a nebulizer including a micro ultrasonic atomizing head, a button cell or a rechargeable lithium battery, and a liquid storage bottle.

In the disclosure, the celery seed, the spring onion seed, the watercress, the crowndaisy Chrysanthemum, and the water hyacinth are all fresh products. The spring onion seed described in the disclosure, also known as watercress, is a perennial aquatic herb in watercress of Cruciferae. The celery seed mentioned in the disclosure is a seed of celery, and the spring onion seed mentioned in the disclosure is a seed of spring onion.

The raw materials of that technical solution of the disclosure are the celery seed, the spring onion seed, the watercress, the crowndaisy Chrysanthemum, the water hyacinth, the zinc oxide and the calcium carbonate, and this composition is obtained through creative experiments, and the follow experiments are result experiments of the disclosure based on many creative experiments.

### Experiment 1

The experiment 1 is a screening experiment, and the screening experiment includes experiment groups 1-3.

Raw materials of the experiment group 1 include: 4000 grams (g) of celery seed, 600 g of spring onion seed, 5000 g of watercress, 4500 g of crowndaisy Chrysanthemum, 2500 g of water hyacinth, 5000 g of monazite, 5000 g of zinc oxide, and 3500 g of calcium carbonate.

A preparation method for the experiment group 1 includes the following steps 1-3:
step 1, crushing the celery seed, the spring onion seed, the watercress, the crowndaisy Chrysanthemum, and the water hyacinth into a slurry; filtering the slurry to remove a residue of the slurry and thus obtain a filtrate; heating and boiling the filtrate for 60 minutes to obtain a heated and boiled filtrate, and filtering the heated and boiled filtrate to obtain a filtered filtrate; adding a fructo-oligosaccharide and an isomaltooligosaccharide into the filtered filtrate, where a weight of the fructo-oligosaccharide and the isomaltooligosaccharide is 0.2 times that of the filtered filtrate, and a weight ratio of the fructo-oligosaccharide to the isomaltooligosaccharide is 1:1; performing uniformly stirring on the filtered filtrate added with the fructo-oligosaccharide and the isomaltooligosaccharide to obtain a stirred filtrate, placing the stirred filtrate in a fermentation tank for a first fermentation to obtain fermentation liquid I, where a fermentation temperature for the first fermentation is 25° C., and a fermentation period for the first fermentation is 16 days; taking out and filtering the fermentation liquid I to obtain a filtrate II, placing the filtrate II in the fermentation tank, and adding the fructo-oligosaccharide in the fermentation tank for a second fermentation to obtain fermentation liquid II, where a weight of the fructo-oligosaccharide for the second fermentation is 0.15 times of a weight of the filtrate II, a fermentation temperature for the second fermentation is 20° C., and a fermentation period for the second fermentation is 18 days; taking out and filtering the fermentation liquid II to obtain a filtrate III, placing the filtrate III in the fermentation tank, adding the isomaltooligosaccharide to the filtrate III for a third fermentation to obtain fermentation liquid III, where a weight of the added isomaltooligosaccharide for the third fermentation is 0.15 times that of the filtrate III, a fermentation temperature for the third fermentation is 20° C., and a fermentation period for the third fermentation is 18 days; and distilling the fermentation liquid III to a distilling temperature of 25° C. with a relative density of 1.20-1.25, and thereby obtaining a fermented product;
step 2, pulverizing the monazite, the calcium carbonate and the zinc oxide and uniformly mixing to obtain a powder; placing the powder in a furnace at a temperature of 2000° C. for firing for 90 minutes to obtain a fired powder; and placing the fired powder in a 1000-mesh cloth bag and performing distilling with a steamer until there is no distillate, to thereby obtain a distilled liquid; and
step 3, completely mixing the fermented product and the distilled liquid to obtain a mixture; and extracting the mixture by using Camellia oil to obtain a Camellia oil layer, where a weight of the Camellia oil is 2.5 times that of the fermented product and the distilled liquid.

Raw materials of the experiment group 2 include: 600 g of spring onion seed, 5000 g of watercress, 4500 g of crowndaisy Chrysanthemum, 5000 g of monazite, 5000 g of zinc oxide, and 3500 g of calcium carbonate.

A preparation method for the experiment group 2 includes the following steps 1-3:
step 1, crushing the spring onion seed, the watercress, and the crowndaisy Chrysanthemum into a slurry; filtering the slurry to remove a residue of the slurry and thus obtain a filtrate; heating and boiling the filtrate for 60 minutes to obtain a heated and boiled filtrate, and filtering the heated and boiled filtrate to obtain a filtered filtrate; adding a fructo-oligosaccharide and an isomaltooligosaccharide into the filtered filtrate, where a weight of the fructo-oligosaccharide and the isomaltooligosaccharide is 0.2 times that of the filtered filtrate, and a weight ratio of the fructo-oligosaccharide to the isomaltooligosaccharide is 1:1; performing uniformly stirring on the filtered filtrate added with the fructo-oligosaccharide and the isomaltooligosaccharide to obtain a stirred filtrate, placing the stirred filtrate in a fermentation tank for a first fermentation to obtain fermentation liquid I, where a fermentation temperature for the first fermentation is 20° C., and a fermentation period for the first fermentation is 18 days; taking out and filtering the fermentation liquid I to obtain a filtrate II, placing the filtrate II in the fermentation tank, and adding the fructo-oligosaccharide in the fermentation tank for a second fermentation to obtain fermentation liquid II, where a weight of the fructo-oligosaccharide for the second fermentation is 0.15 times of a weight of the filtrate II, a fermentation temperature for the second fermentation is 20° C., and a fermentation period for the second fermentation is 18 days; taking out and filtering the fermentation liquid II to obtain a filtrate III, placing the filtrate III in the fermentation tank, and adding the isomaltooligosaccharide to the filtrate III for a third fermentation to obtain fermentation liquid III, where a weight of the added isomaltooligosaccharide for the third fermentation is 0.15 times that of the filtrate III, a fermentation temperature for the third fermentation is 20° C., and a fermentation period for the third fermentation is 18 days; and distilling the fermentation liquid III to a distilling temperature of 25° C. with a relative density of 1.20-1.25, and thereby obtaining a fermented product;
step 2, pulverizing the monazite, the calcium carbonate and the zinc oxide and uniformly mixing to obtain a powder; placing the powder in a furnace at a temperature of 2000° C. for firing for 90 minutes to obtain a fired powder; and placing the fired powder in a 1000-mesh cloth bag and performing distilling with a steamer until there is no distillate, to thereby obtain a distilled liquid; and
step 3, completely mixing the fermented product and the distilled liquid to obtain a mixture; and extracting the mixture by using Camellia oil to obtain a Camellia oil layer, where a weight of the Camellia oil is 2.5 times that of the fermented product and the distilled liquid.

Raw materials of the experiment group 3 include: 4000 g of celery seed, 600 g of spring onion seed, 5000 g of watercress, 4500 g of crowndaisy Chrysanthemum, 2500 g of water hyacinth, and 5000 g of monazite.

A preparation method for the experiment group 3 includes the following steps 1-3:
step 1, crushing the celery seed, the spring onion seed, the watercress, the crowndaisy Chrysanthemum, and the water hyacinth into a slurry; filtering the slurry to remove a residue of the slurry and thus obtain a filtrate; heating and boiling the filtrate for 60 minutes to obtain a heated and boiled filtrate, and filtering the heated and boiled filtrate to obtain a filtered filtrate; adding a fructo-oligosaccharide and an isomaltooligosaccharide into the filtered filtrate, where a weight of the fructo-oligosaccharide and the isomaltooligosaccharide is 0.2 times that of the filtered filtrate, and a weight ratio of the fructo-oligosaccharide to the isomaltooligosaccharide is 1:1; performing uniformly stirring on the filtered filtrate added with the fructo-oligosaccharide and the isomaltooligosaccharide to obtain a stirred filtrate, placing the stirred filtrate in a fermentation tank for a first fermentation to obtain fermentation liquid I, where a fermentation temperature for the first fermentation is 20° C., and a fermentation period for the first fermentation is 18 days; taking out and filtering the fermentation liquid I to obtain a filtrate II, placing the filtrate II in the fermentation tank, and adding the fructo-oligosaccharide in the fermentation tank for a second fermentation to obtain fermentation liquid II, where a weight of the fructo-oligosaccharide for the second fermentation is 0.15 times of a weight of the filtrate II, a fermentation temperature for the second fermentation is 20° C., and a fermentation period for the second fermentation is 18 days; taking out and filtering the fermentation liquid II to obtain a filtrate III, placing the filtrate III in the fermentation tank, adding the isomaltooligosaccharide to the filtrate III for a third fermentation to obtain fermentation liquid III, where a weight of the added isomaltooligosaccharide for the third fermentation is 0.15 times that of the filtrate III, a fermentation temperature for the third fermentation is 20° C., and a fermentation period for the third fermentation is 18 days; and distilling the fermentation liquid III to a distilling temperature of 25° C. with a relative density of 1.20-1.25, and thereby obtaining a fermented product;
step 2, pulverizing the monazite to obtain a powder; placing the powder in a furnace at a temperature of 2000° C. for firing for 90 minutes to obtain a fired powder; and placing the fired powder in a 1000-mesh cloth bag and performing distilling with a steamer until there is no distillate, to thereby obtain a distilled liquid; and
step 3, completely mixing the fermented product and the distilled liquid to obtain a mixture; and extracting the mixture by using Camellia oil to obtain a Camellia oil layer, where a weight of the Camellia oil is 2.5 times that of the fermented product and the distilled liquid.

A test method is performed.

Kunming mice are used, each of the Kunming mice is 18-22 g in weight, and there is no restriction on sexes of the Kunming mice (provided by Pharmacology Department of Traditional Chinese Medicine, Beijing University of Chinese Medicine).

The test method includes: randomly dividing the Kunming mice into a control group and test groups 1-3; administering physiological saline to the control group by intragastric administration with a dosage of 25 milligrams per kilogram (mg/kg) once a day for 7 days; administering Camellia oil layers corresponding to the experimental groups 1-3 to the test groups 1-3 by intragastric administration with a dosage of 1.60 grams per kilogram (g/kg) once a day for 7 days; on the 7-th day, measuring left foot volumes of the kunming mice of the control group and the test groups 1-3 by using a mouse toe volume measuring instrument to obtain normal values of the left foot volumes before administration, administering corresponding drugs (i.e., the physiological saline and the Camellia oil layers) to the control group and the test groups 1-3, and one hour after the administering, performing intradermal injection on foot bottoms of the control group and the test groups 1-3 with 0.03 milliliters (ml) of carrageenan with a concentration of 1%; and after 3 hours of the intradermal injection, measuring left hind foot volumes of the control group and the test groups 1-3 to obtain swelling volumes of left hind feet of the control group and the test groups 1-3 after administration, and calculating swelling rates of the left hind feet of the control group and the test groups 1-3.

Test results are shown in Table 1.

TABLE 1
Effects of different groups on swelling rates of feet of mice

| | a number | swelling rate 3 hours after |
|---|---|---|
| Group | of animals | inflammation (%) |
| Control group | 10 | 85.4 ± 7.4 |
| Test group 1 | 10 | 41.7 ± 6.9** |
| Test group 2 | 10 | 63.9 ± 5.4* |
| Test group 3 | 10 | 82.9 ± 9.1 |

Note:
Compared with the control group,
**P < 0.01, and
*P < 0.05.

An experimental conclusion is obtained as follows. The above experiment shows that when the celery seed and the water hyacinth are removed from the raw materials, the prepared Camellia oil layer has a certain effect on inhibiting foot swelling in mice, which is significantly different from that in the control group; when the zinc oxide and calcium carbonate are removed from the raw materials, an effect of the prepared Camellia oil layer on inhibiting foot swelling in mice is decreased, which is not statistically significant compared with the control group. However, the prepared Camellia oil layer from the raw materials in the disclosure has a significant effect on inhibiting foot swelling in mice, with a very significant difference compared with the control group (P<0.01). This fully shows that the composition of the raw materials of the disclosure is an organic whole, the whole system is playing a pharmacological role, and any one of the raw materials of the disclosure is indispensable.

### DETAILED DESCRIPTION OF EMBODIMENTS

In order to make objectives, technical solutions and advantages of the disclosure more clear, the disclosure will be further described in detail with reference to specific embodiments. It should be understood that these descriptions are merely exemplary and are not intended to limit the scope of the disclosure. In addition, in the following description, descriptions of well-known structures and technologies are omitted to avoid unnecessarily confusing the concepts of the disclosure.

### Embodiment 1

Raw materials of nebulized inhalation liquid include 3000 g of celery seed, 400 g of spring onion seed, 4000 g of watercress, 3500 g of crowndaisy Chrysanthemum, 1500 g of water hyacinth, 4000 g of monazite, 4000 g of zinc oxide, and 3000 g of calcium carbonate.

A preparation method of the nebulized inhalation liquid includes the following steps 1-3:
step 1, crushing the celery seed, the spring onion seed, the watercress, the crowndaisy Chrysanthemum, and the water hyacinth into a slurry; filtering the slurry to remove a residue of the slurry and thus obtain a filtrate; heating and boiling the filtrate for 50 minutes to obtain a heated and boiled filtrate, and filtering the heated and boiled filtrate to obtain a filtered filtrate; adding a fructo-oligosaccharide and an isomaltooligosaccharide into the filtered filtrate, where a weight of the fructo-oligosaccharide and the isomaltooligosaccharide is 0.3 times that of the filtered filtrate, and a weight ratio of the fructo-oligosaccharide to the isomaltooligosaccharide is 1:1; performing uniformly stirring on the filtered filtrate added with the fructo-oligosaccharide and the isomaltooligosaccharide to obtain a stirred filtrate, placing the stirred filtrate in a fermentation tank for a first fermentation to obtain fermentation liquid I, where a fermentation temperature for the first fermentation is 25° C., and a fermentation period for the first fermentation is 16 days; taking out and filtering the fermentation liquid I to obtain a filtrate II, placing the filtrate II in the fermentation tank, and adding the fructo-oligosaccharide in the fermentation tank for a second fermentation to obtain fermentation liquid II, where a weight of the fructo-oligosaccharide for the second fermentation is 0.2 times of a weight of the filtrate II, a fermentation temperature for the second fermentation is 25° C., and a fermentation period for the second fermentation is 16 days; taking out and filtering the fermentation liquid II to obtain a filtrate III, placing the filtrate III in the fermentation tank, adding the isomaltooligosaccharide to the filtrate III for a third fermentation to obtain fermentation liquid III, where a weight of the added isomaltooligosaccharide for the third fermentation is 0.2 times that of the filtrate III, a fermentation temperature for the third fermentation is 25° C., and a fermentation period for the third fermentation is 24 days; and distilling the fermentation liquid III to a distilling temperature of 25° C. with a relative density of 1.20-1.25, and thereby obtaining a fermented product;
step 2, pulverizing the monazite, the calcium carbonate and the zinc oxide and uniformly mixing to obtain a powder; placing the powder in a furnace at a temperature of 1800° C. for firing for 100 minutes to obtain a fired powder; and placing the fired powder in a 1000-mesh cloth bag and performing distilling with a steamer until there is no distillate, to thereby obtain a distilled liquid; and
step 3, completely mixing the fermented product and the distilled liquid to obtain a mixture; extracting the mixture by using Camellia oil to obtain a Camellia oil layer, where a weight of the Camellia oil is 2 times that of the fermented product and the distilled liquid; and adding glycerol (C₃H₈O₃), 1,2-propylene glycol (C₃H₈O₂) and structured water into the Camellia oil layer to obtain the nebulized inhalation liquid, where the glycerol is 300 g, the 1,2-propylene glycol is 200 g, and the structured water is 300 g. The structured water is also referred to small molecular group water, micro cluster water, small cluster water, or micro clustered water; and within 100 hertz (Hz), the structured water is composed of 5-7 water molecules.

### Embodiment 2

Raw materials of nebulized inhalation liquid include 5000 g of celery seed, 800 g of spring onion seed, 6000 g of watercress, 5500 g of crowndaisy Chrysanthemum, 3500 g of water hyacinth, 6000 g of monazite, 6000 g of zinc oxide, and 4000 g of calcium carbonate.

A preparation method of the nebulized inhalation liquid includes the following steps 1-3:
step 1, crushing the celery seed, the spring onion seed, the watercress, the crowndaisy Chrysanthemum, and the water hyacinth into a slurry; filtering the slurry to remove a residue of the slurry and thus obtain a filtrate; heating and boiling the filtrate for 70 minutes to obtain a heated and boiled filtrate, and filtering the heated and boiled filtrate to obtain a filtered filtrate; adding a fructo-oligosaccharide and an isomaltooligosaccharide into the filtered filtrate, where a weight of the fructo-oligosaccharide and the isomaltooligosaccharide is 0.1 times that of the filtered filtrate, and a weight ratio of the fructo-oligosaccharide to the isomaltooligosaccharide is 1:1; performing uniformly stirring on the filtered filtrate added with the fructo-oligosaccharide and the isomaltooligosaccharide to obtain a stirred filtrate, placing the stirred filtrate in a fermentation tank for a first fermentation to obtain fermentation liquid I, where a fermentation temperature for the first fermentation is 18° C., and a fermentation period for the first fermentation is 24 days; taking out and filtering the fermentation liquid I to obtain a filtrate II, placing the filtrate II in the fermentation tank, and adding the fructo-oligosaccharide in the fermentation tank for a second fermentation to obtain fermentation liquid II, where a weight of the fructo-oligosaccharide for the second fermentation is 0.1 times of a weight of the filtrate II, a fermentation temperature for the second fermentation is 18° C., and a fermentation period for the second fermentation is 24 days; taking out and filtering the fermentation liquid II to obtain a filtrate III, placing the filtrate III in the fermentation tank, adding the isomaltooligosaccharide to the filtrate III for a third fermentation to obtain fermentation liquid III, where a weight of the added isomaltooligosaccharide for the third fermentation is 0.1 times that of the filtrate III, a fermentation temperature for the third fermentation is 18° C., and a fermentation period for the third fermentation is 24 days; and distilling the fermentation liquid III to a distilling temperature of 25° C. with a relative density of 1.20-1.25, and thereby obtaining a fermented product;
step 2, pulverizing the monazite, the calcium carbonate and the zinc oxide and uniformly mixing to obtain a powder; placing the powder in a furnace at a temperature of 2200° C. for firing for 80 minutes to obtain a fired powder; and placing the fired powder in a 1000-mesh cloth bag and performing distilling with a steamer until there is no distillate, to thereby obtain a distilled liquid; and
step 3, completely mixing the fermented product and the distilled liquid to obtain a mixture; extracting the mixture by using Camellia oil to obtain a Camellia oil layer, where a weight of the Camellia oil is 2-3 times that of the fermented product and the distilled liquid; and adding glycerol (C₃H₈O₃), 1,2-propylene glycol (C₃H₈O₂) and structured water into the Camellia oil layer to obtain the nebulized inhalation liquid, where the glycerol is 400 g, the 1,2-propylene glycol is 300 g, and the structured water is 600 g.

### Embodiment 3

Raw materials of nebulized inhalation liquid include 4000 g of celery seed, 600 g of spring onion seed, 5000 g of watercress, 4500 g of crowndaisy Chrysanthemum, 2500 g of water hyacinth, 5000 g of monazite, 5000 g of zinc oxide, and 3500 g of calcium carbonate.

A preparation method of the nebulized inhalation liquid includes the following steps 1-3:
step 1, crushing the celery seed, the spring onion seed, the watercress, the crowndaisy Chrysanthemum, and the water hyacinth into a slurry; filtering the slurry to remove a residue of the slurry and thus obtain a filtrate; heating and boiling the filtrate for 60 minutes to obtain a heated and boiled filtrate, and filtering the heated and boiled filtrate to obtain a filtered filtrate; adding a fructo-oligosaccharide and an isomaltooligosaccharide into the filtered filtrate, where a weight of the fructo-oligosaccharide and the isomaltooligosaccharide is 0.2 times that of the filtered filtrate, and a weight ratio of the fructo-oligosaccharide to the isomaltooligosaccharide is 1:1; performing uniformly stirring on the filtered filtrate added with the fructo-oligosaccharide and the isomaltooligosaccharide to obtain a stirred filtrate, placing the stirred filtrate in a fermentation tank for a first fermentation to obtain fermentation liquid I, where a fermentation temperature for the first fermentation is 20° C., and a fermentation period for the first fermentation is 18 days; taking out and filtering the fermentation liquid I to obtain a filtrate II, placing the filtrate II in the fermentation tank, and adding the fructo-oligosaccharide in the fermentation tank for a second fermentation to obtain fermentation liquid II, where a weight of the fructo-oligosaccharide for the second fermentation is 0.15 times of a weight of the filtrate II, a fermentation temperature for the second fermentation is 20° C., and a fermentation period for the second fermentation is 18 days; taking out and filtering the fermentation liquid II to obtain a filtrate III, placing the filtrate III in the fermentation tank, adding the isomaltooligosaccharide to the filtrate III for a third fermentation to obtain fermentation liquid III, where a weight of the added isomaltooligosaccharide for the third fermentation is 0.15 times that of the filtrate III, a fermentation temperature for the third fermentation is 20° C., and a fermentation period for the third fermentation is 18 days; and distilling the fermentation liquid III to a distilling temperature of 25° C. with a relative density of 1.20-1.25, and thereby obtain a fermented product;
step 2, pulverizing the monazite, the calcium carbonate and the zinc oxide and uniformly mixing to obtain a powder; placing the powder in a furnace at a temperature of 2000° C. for firing for 90 minutes to obtain a fired powder; and placing the fired powder in a 1000-mesh cloth bag and performing distilling with a steamer until there is no distillate, to thereby obtain a distilled liquid; and
step 3, completely mixing the fermented product and the distilled liquid to obtain a mixture; extracting the mixture by using Camellia oil to obtain a Camellia oil layer, where a weight of the Camellia oil is 2.5 times that of the fermented product and the distilled liquid; and adding glycerol (C₃H₈O₃), 1,2-propylene glycol (C₃H₈O₂) and structured water into the Camellia oil layer to obtain the nebulized inhalation liquid, where the glycerol is 350 g, the 1,2-propylene glycol is 250 g, and the structured water is 450 g.

### Embodiment 4

Raw materials of nebulized inhalation liquid include 3500 g of celery seed, 500 g of spring onion seed, 4500 g of watercress, 4000 g of crowndaisy Chrysanthemum, 2000 g of water hyacinth, 4500 g of monazite, 4500 g of zinc oxide, and 3300 g of calcium carbonate.

A preparation method of the nebulized inhalation liquid includes the following steps 1-3:
step 1, crushing the celery seed, the spring onion seed, the watercress, the crowndaisy Chrysanthemum, and the water hyacinth into a slurry; filtering the slurry to remove a residue of the slurry and thus obtain a filtrate; heating and boiling the filtrate for 55 minutes to obtain a heated and boiled filtrate, and filtering the heated and boiled filtrate to obtain a filtered filtrate; adding a fructo-oligosaccharide and an isomaltooligosaccharide into the filtered filtrate, where a weight of the fructo-oligosaccharide and the isomaltooligosaccharide is 0.15 times that of the filtered filtrate, and a weight ratio of the fructo-oligosaccharide to the isomaltooligosaccharide is 1:1; performing uniformly stirring on the filtered filtrate added with the fructo-oligosaccharide and the isomaltooligosaccharide to obtain a stirred filtrate, placing the stirred filtrate in a fermentation tank for a first fermentation to obtain fermentation liquid I, where a fermentation temperature for the first fermentation is 22° C., and a fermentation period for the first fermentation is 22 days; taking out and filtering the fermentation liquid I to obtain a filtrate II, placing the filtrate II in the fermentation tank, and adding the fructo-oligosaccharide in the fermentation tank for a second fermentation to obtain fermentation liquid II, where a weight of the fructo-oligosaccharide for the second fermentation is 0.15 times of a weight of the filtrate II, a fermentation temperature for the second fermentation is 22° C., and a fermentation period for the second fermentation is 22 days; taking out and filtering the fermentation liquid II to obtain a filtrate III, placing the filtrate III in the fermentation tank, adding the isomaltooligosaccharide to the filtrate III for a third fermentation to obtain fermentation liquid III, where a weight of the added isomaltooligosaccharide for the third fermentation is 0.15 times that of the filtrate III, a fermentation temperature for the third fermentation is 22° C., and a fermentation period for the third fermentation is 22 days; and distilling the fermentation liquid III to a distilling temperature of 25° C. with a relative density of 1.20-1.25, and thereby obtain a fermented product;
step 2, pulverizing the monazite, the calcium carbonate and the zinc oxide and uniformly mixing to obtain a powder; placing the powder in a furnace at a temperature of 2100° C. for firing for 85 minutes to obtain a fired powder; and placing the fired powder in a 1000-mesh cloth bag and performing distilling with a steamer until there is no distillate, to thereby obtain a distilled liquid; and
step 3, completely mixing the fermented product and the distilled liquid to obtain a mixture; extracting the mixture by using Camellia oil to obtain a Camellia oil layer, where a weight of the Camellia oil is 2 times that of the fermented product and the distilled liquid; and adding glycerol (C₃H₈O₃), 1,2-propylene glycol (C₃H₈O₂) and structured water into the Camellia oil layer to obtain the nebulized inhalation liquid, where the glycerol is 320 g, the 1,2-propylene glycol is 235 g, and the structured water is 400 g.

### Embodiment 5

Raw materials of nebulized inhalation liquid include 4500 g of celery seed, 750 g of spring onion seed, 5500 g of watercress, 5200 g of crowndaisy Chrysanthemum, 3300 g of water hyacinth, 5800 g of monazite, 5800 g of zinc oxide, and 3800 g of calcium carbonate.

A preparation method of the nebulized inhalation liquid includes the following steps 1-3:
step 1, crushing the celery seed, the spring onion seed, the watercress, the crowndaisy Chrysanthemum, and the water hyacinth into a slurry; filtering the slurry to remove a residue of the slurry and thus obtain a filtrate; heating and boiling the filtrate for 65 minutes to obtain a heated and boiled filtrate, and filtering the heated and boiled filtrate to obtain a filtered filtrate; adding a fructo-oligosaccharide and an isomaltooligosaccharide into the filtered filtrate, where a weight of the fructo-oligosaccharide and the isomaltooligosaccharide is 0.25 times that of the filtered filtrate, and a weight ratio of the fructo-oligosaccharide to the isomaltooligosaccharide is 1:1; performing uniformly stirring on the filtered filtrate added with the fructo-oligosaccharide and the isomaltooligosaccharide to obtain a stirred filtrate, placing the stirred filtrate in a fermentation tank for a first fermentation to obtain fermentation liquid I, where a fermentation temperature for the first fermentation is 20° C., and a fermentation period for the first fermentation is 18 days; taking out and filtering the fermentation liquid I to obtain a filtrate II, placing the filtrate II in the fermentation tank, and adding the fructo-oligosaccharide in the fermentation tank for a second fermentation to obtain fermentation liquid II, where a weight of the fructo-oligosaccharide for the second fermentation is 0.15 times of a weight of the filtrate II, a fermentation temperature for the second fermentation is 20° C., and a fermentation period for the second fermentation is 20 days; taking out and filtering the fermentation liquid II to obtain a filtrate III, placing the filtrate III in the fermentation tank, adding the isomaltooligosaccharide to the filtrate III for a third fermentation to obtain fermentation liquid III, where a weight of the added isomaltooligosaccharide for the third fermentation is 0.15 times that of the filtrate III, a fermentation temperature for the third fermentation is 20° C., and a fermentation period for the third fermentation is 20 days; and distilling the fermentation liquid III to a distilling temperature of 25° C. with a relative density of 1.20-1.25, and thereby obtain a fermented product;
   step 2, pulverizing the monazite, the calcium carbonate and the zinc oxide and uniformly mixing to obtain a powder; placing the powder in a furnace at a temperature of 2000° C. for firing for 95 minutes to obtain a fired powder; and placing the fired powder in a 1000-mesh cloth bag and performing distilling with a steamer until there is no distillate, to thereby obtain a distilled liquid; and
step 3, completely mixing the fermented product and the distilled liquid to obtain a mixture; extracting the mixture by using Camellia oil to obtain a Camellia oil layer, where a weight of the Camellia oil is 2-3 times that of the fermented product and the distilled liquid; and adding glycerol (C₃H₈O₃), 1,2-propylene glycol (C₃H₈O₂) and structured water into the Camellia oil layer to obtain the nebulized inhalation liquid, where the glycerol is 380 g, the 1,2-propylene glycol is 285 g, and the structured water is 550 g.

In the disclosure, the celery seed, the spring onion seed, the watercress, the crowndaisy Chrysanthemum, and the water hyacinth are all fresh products.

The nebulized inhalation liquid can be applied to a medical nebulizer in a prior art for patients with rhinitis.

The nebulized inhalation liquid can also be applied to a nebulizer similar to a nebulizer including a micro ultrasonic atomizing head, a button cell or a rechargeable lithium battery, and a liquid storage bottle. Specifically, the nebulized inhalation liquid is contained in the liquid storage bottle.

### Experiment 2

Effects on mice with rhinitis are obtained through the experiment 2.

The nebulized inhalation liquid prepared in the embodiment 3 is applied to a test group 1.

The nebulized inhalation liquid prepared in the embodiment 4 is applied to a test group 2.

Male BALB/c mice are used, each of the male BALB/c mice is 16-20 g in weight (provided by Pharmacology Department of Traditional Chinese Medicine, Beijing University of Chinese Medicine). During the experiment 2, the following steps are performed: randomly dividing the male BALB/c mice into groups including a blank control group, a model group, and the test groups 1-2; on a first day, performing intraperitoneal injection on the model group and the test groups 1-2 by using 200 microlitres (μL) of a physiological saline suspension containing 50 micrograms (μg) of ovalbumin and 2 milligrams (mg) of an aluminum hydroxide adjuvant powder; on an eighth day, performing intraperitoneal injection on the model group and the test groups 1-2 by using 200 μL of a physiological saline suspension containing 50 μg of ovalbumin and 2 mg of an aluminum hydroxide adjuvant powder; on a fifteenth day, performing intraperitoneal injection on the model group and the test groups 1-2 by using 200 μL of a physiological saline suspension containing 50 μg of ovalbumin and 2 mg of an aluminum hydroxide adjuvant powder; and after the intraperitoneal injection on the fifteenth day, another week later, from a 22nd day, dripping an ovalbumin physiological saline solution with a concentration of 5% into noses of the model group and the test groups 1-2 once a day, with 10 μL of the ovalbumin physiological saline solution for each nasal cavity. For the blank control group, during basic sensitization of abdominal cavity and nasal stimulation (i.e., the above intraperitoneal injection and dripping), physiological saline is fed to the blank group. From a 29th day, the nebulized inhalation liquids prepared in the embodiments 3-4 are respectively fed into nasal cavities of the experiment groups 1-2 through nasal administration, with a dosage of 3 g/kg, once a day for 14 consecutive days; vaseline is applied to noses of the model group through nasal administration once a day for 14 consecutive days; and physiological saline is fed for the blank control group through nasal administration once a day for 14 consecutive days. The mice of the blank control group, the model group, and the test groups 1-2 are killed 24 hours after the last nasal administration. Then, nasal lavage fluids are obtained, kept standing, centrifuged, sub-packed, and finally stored at a temperature of −80° C. A relevant instruction of a kit is referred, and expression levels of Interleukin-2 (IL-2), Interleukin-4 (IL-4), Interleukin-5 (IL-5) and Interferon gamma (IFN-γ) in the nasal lavage fluids are detected. It is noted that, the above methods are carried out with reference to literature methods.

Experimental results are shown in Table 2.

TABLE 2
Effects on mice with rhinitis

| | | IL-2 | | | |
|---|---|---|---|---|---|
| | | (picograms | | | |
| | a number | per milliliter | | | |
| Group | of animals | (pg/mL)) | IFN-γ(pg/mL) | IL-4 (pg/mL) | IL-5 (pg/mL) |
| Blank control | 10 | 49.19 ± 3.74** | 80.62 ± 5.39** | 14.71 ± 1.28** | 26.71 ± 2.85** |
| group | | | | | |
| Model group | 10 | 28.92 ± 3.18 | 43.17 ± 3.92 | 38.56 ± 2.97 | 51.34 ± 4.67 |
| Test group 1 | 10 | 38.77 ± 4.21* | 69.44 ± 5.29* | 27.19 ± 2.38* | 39.78 ± 3.47* |
| Test group 2 | 10 | 37.69 ± 3.27* | 66.28 ± 5.11* | 30.28 ± 2.70* | 41.29 ± 3.72* |

Note:
Compared with the model group,
**P < 0.01, and
*P < 0.05.

An experimental conclusion is obtained as follows. The above experiment shows that the product prepared from the raw materials of the disclosure has a good therapeutic effect on mice with rhinitis, and there is a significant difference compared with the model group, which shows that the nebulized inhalation liquids prepared in the disclosure can alleviate rhinitis and has an auxiliary therapeutic effect on rhinitis.

The above is merely the illustrated embodiment of the disclosure, and it should be pointed out that a person skilled in the art can make several improvements and embellishments without departing from the principle of the disclosure, and these improvements and embellishments should also be regarded as the protection scope of the disclosure.

What is claimed is:

1. Nebulized inhalation liquid for relieving rhinitis, wherein raw materials of the nebulized inhalation liquid comprise: celery seed, spring onion seed, watercress, crowndaisy Chrysanthemum, water hyacinth, monazite, zinc oxide and calcium carbonate.

2. The nebulized inhalation liquid for relieving rhinitis as claimed in claim 1, wherein the celery seed is 300-500 parts by weight, the spring onion seed is 40-80 parts by weight, the watercress is 400-600 parts by weight, the crowndaisy Chrysanthemum is 350-550 parts by weight, the water hyacinth is 150-350 parts by weight, the monazite is 400-600 parts by weight, the zinc oxide is 400-600 parts by weight, and the calcium carbonate is 300-400 parts by weight.

3. The nebulized inhalation liquid for relieving rhinitis as claimed in claim 1, wherein the celery seed is 400 parts by weight, the spring onion seed is 60 parts by weight, the watercress is 500 parts by weight, the crowndaisy Chrysanthemum is 450 parts by weight, the water hyacinth is 250 parts by weight, the monazite is 500 parts by weight, the zinc oxide is 500 parts by weight, and the calcium carbonate is 350 parts by weight.

4. The nebulized inhalation liquid for relieving rhinitis as claimed in claim 1, wherein the nebulized inhalation liquid is applied to prepare a medicine for adjuvant treatment of rhinitis.

5. The nebulized inhalation liquid for relieving rhinitis as claimed in claim 2, wherein the nebulized inhalation liquid is applied to prepare a medicine for adjuvant treatment of rhinitis.

6. The nebulized inhalation liquid for relieving rhinitis as claimed in claim 3, wherein the nebulized inhalation liquid is applied to prepare a medicine for adjuvant treatment of rhinitis.

7. The nebulized inhalation liquid for relieving rhinitis as claimed in claim 1, wherein a preparation method of the nebulized inhalation liquid comprises:
step 1, crushing the celery seed, the spring onion seed, the watercress, the crowndaisy Chrysanthemum, and the water hyacinth into a slurry; filtering the slurry to remove a residue of the slurry and thus obtain a filtrate; heating and boiling the filtrate for 50-70 minutes to obtain a heated and boiled filtrate, and filtering the heated and boiled filtrate to obtain a filtered filtrate; adding a fructo-oligosaccharide (FOS) and an isomaltooligosaccharide (IMO) into the filtered filtrate, wherein a weight of the FOS and the IMO is 0.1-0.3 times that of the filtered filtrate, and a weight ratio of the FOS to the IMO is 1:1; performing uniformly stirring on the filtered filtrate added with the FOS and the IMO to obtain a stirred filtrate, placing the stirred filtrate in a fermentation tank for a first fermentation to obtain fermentation liquid I, wherein a fermentation temperature for the first fermentation is in a range of 18 degrees Celsius (° C.)-25° C., and a fermentation period for the first fermentation is in a range of 16-24 days; taking out and filtering the fermentation liquid I to obtain a filtrate II, placing the filtrate II in the fermentation tank, and adding a FOS in the fermentation tank for a second fermentation to obtain fermentation liquid II, wherein a weight of the FOS for the second fermentation is 0.1-0.2 times of a weight of the filtrate II, a fermentation temperature for the second fermentation is 18° C.-25° C., and a fermentation period for the second fermentation is 16-24 days; taking out and filtering the fermentation liquid II to obtain a filtrate III, placing the filtrate III in the fermentation tank, adding an IMO to the filtrate III for a third fermentation to obtain fermentation liquid III, wherein a weight of the IMO for the third fermentation is 0.1-0.2 times that of the filtrate III, a fermentation temperature for the third fermentation is 18° C.-25° C., and a fermentation period for the third fermentation is 16-24 days; and distilling the fermentation liquid III to a distilling temperature of 25° C. with a relative density of 1.20-1.25, and thereby obtaining a fermented product;
step 2, pulverizing the monazite, the calcium carbonate and the zinc oxide and uniformly mixing to obtain a powder; placing the powder in a furnace at a temperature of 1800° C.-2200° C. for firing for 80-100 minutes to obtain a fired powder; and placing the fired powder in a 1000-mesh cloth bag and performing distilling with a steamer until there is no distillate, to thereby obtain a distilled liquid; and
step 3, completely mixing the fermented product and the distilled liquid to obtain a mixture; extracting the mixture by using Camellia oil to obtain a Camellia oil layer, wherein a weight of the Camellia oil is 2-3 times that of the fermented product and the distilled liquid; and adding glycerol (C₃H₈O₃), 1,2-propylene glycol (C₃H₈O₂) and structured water into the Camellia oil layer to obtain the nebulized inhalation liquid.

8. The nebulized inhalation liquid for relieving rhinitis as claimed in claim 7, wherein the glycerol is 30-40 parts by weight, the 1,2-propylene glycol is 20-30 parts by weight, and the structured water is 30-60 parts by weight.

9. The nebulized inhalation liquid for relieving rhinitis as claimed in claim 7, wherein the glycerol is 35 parts by weight, the 1,2-propylene glycol is 25 parts by weight, and the structured water is 45 parts by weight.

10. The nebulized inhalation liquid for relieving rhinitis according to claim 1, wherein the nebulized inhalation liquid is applied to a nebulizer, and the nebulizer comprises a micro ultrasonic atomizing head, a button cell or a rechargeable lithium battery, and a liquid storage bottle.

11. A preparation method of the nebulized inhalation liquid as claimed in claim 1, comprising:
step 1, crushing the celery seed, the spring onion seed, the watercress, the crowndaisy Chrysanthemum, and the water hyacinth into a slurry; filtering the slurry to remove a residue of the slurry and thus obtain a filtrate; heating and boiling the filtrate for 50-70 minutes to obtain a heated and boiled filtrate, and filtering the heated and boiled filtrate to obtain a filtered filtrate; adding a FOS and an IMO into the filtered filtrate, wherein a weight of the FOS and the IMO is 0.1-0.3 times that of the filtered filtrate, and a weight ratio of the FOS to the IMO is 1:1; performing uniformly stirring on the filtered filtrate added with the FOS and the IMO to obtain a stirred filtrate, placing the stirred filtrate in a fermentation tank for a first fermentation to obtain fermentation liquid I, wherein a fermentation temperature for the first fermentation is in a range of 18 degrees Celsius (° C.)-25° C., and a fermentation period for the first fermentation is in a range of 16-24 days; taking out and filtering the fermentation liquid I to obtain a filtrate II, placing the filtrate II in the fermentation tank, and adding a FOS in the fermentation tank for a second fermentation to obtain fermentation liquid II, wherein a weight of the FOS for the second fermentation is 0.1-0.2 times of a weight of the filtrate II, a fermentation temperature for the second fermentation is 18° C.-25° C., and a fermentation period for the second fermentation is 16-24 days; taking out and filtering the fermentation liquid II to obtain a filtrate III, placing the filtrate III in the fermentation tank, adding an IMO to the filtrate III for a third fermentation to obtain fermentation liquid III, wherein a weight of the IMO for the third fermentation is 0.1-0.2 times that of the filtrate III, a fermentation temperature for the third fermentation is 18° C.-25° C., and a fermentation period for the third fermentation is 16-24 days; and distilling the fermentation liquid III to a distilling temperature of 25° C. with a relative density of 1.20-1.25, and thereby obtaining a fermented product;
step 2, pulverizing the monazite, the calcium carbonate and the zinc oxide and uniformly mixing to obtain a powder; placing the powder in a furnace at a temperature of 1800° C.-2200° C. for firing for 80-100 minutes to obtain a fired powder; and placing the fired powder in a 1000-mesh cloth bag and performing distilling with a steamer until there is no distillate, to thereby obtain a distilled liquid; and
step 3, completely mixing the fermented product and the distilled liquid to obtain a mixture; extracting the mixture by using Camellia oil to obtain a Camellia oil layer, wherein a weight of the Camellia oil is 2-3 times that of the fermented product and the distilled liquid; and adding glycerol (C₃H₈O₃), 1,2-propylene glycol (C₃H₈O₂) and structured water into the Camellia oil layer to obtain the nebulized inhalation liquid.

12. The preparation method of the nebulized inhalation liquid as claimed in claim 11, wherein the celery seed is 300-500 parts by weight, the spring onion seed is 40-80 parts by weight, the watercress is 400-600 parts by weight, the crowndaisy Chrysanthemum is 350-550 parts by weight, the water hyacinth is 150-350 parts by weight, the monazite is 400-600 parts by weight, the zinc oxide is 400-600 parts by weight, and the calcium carbonate is 300-400 parts by weight.

13. The preparation method of the nebulized inhalation liquid as claimed in claim 11, wherein the celery seed is 400 parts by weight, the spring onion seed is 60 parts by weight, the watercress is 500 parts by weight, the crowndaisy Chrysanthemum is 450 parts by weight, the water hyacinth is 250 parts by weight, the monazite is 500 parts by weight, the zinc oxide is 500 parts by weight, and the calcium carbonate is 350 parts by weight.

14. The preparation method of the nebulized inhalation liquid as claimed in claim 11, wherein the glycerol is 30-40 parts by weight, the 1,2-propylene glycol is 20-30 parts by weight, and the structured water is 30-60 parts by weight.

15. The preparation method of the nebulized inhalation liquid as claimed in claim 11, wherein the glycerol is 35 parts by weight, the 1,2-propylene glycol is 25 parts by weight, and the structured water is 45 parts by weight.

16. A nebulizer, comprising a micro ultrasonic atomizing head, a button cell or a rechargeable lithium battery, and a liquid storage bottle;
wherein the liquid storage bottle is configured to store nebulized inhalation liquid, and raw materials of the nebulized inhalation liquid comprise: celery seed, spring onion seed, watercress, crowndaisy Chrysanthemum, water hyacinth, monazite, zinc oxide and calcium carbonate.

17. The nebulizer as claimed in claim 16, wherein the celery seed, the spring onion seed, the watercress, the crowndaisy Chrysanthemum, and the water hyacinth are fresh products; and
wherein the celery seed is 300-500 parts by weight, the spring onion seed is 40-80 parts by weight, the watercress is 400-600 parts by weight, the crowndaisy Chrysanthemum is 350-550 parts by weight, the water hyacinth is 150-350 parts by weight, the monazite is 400-600 parts by weight, the zinc oxide is 400-600 parts by weight, and the calcium carbonate is 300-400 parts by weight.

18. The nebulizer as claimed in claim 16, wherein the celery seed is 400 parts by weight, the spring onion seed is 60 parts by weight, the watercress is 500 parts by weight, the crowndaisy Chrysanthemum is 450 parts by weight, the water hyacinth is 250 parts by weight, the monazite is 500 parts by weight, the zinc oxide is 500 parts by weight, and the calcium carbonate is 350 parts by weight.

19. Nebulized inhalation liquid for relieving rhinitis, comprising: a Camellia oil layer, glycerol, 1,2-propylene glycol, and structured water; and raw materials of the Camellia oil later comprise: Camellia oil, celery seed, spring onion seed, watercress, crowndaisy Chrysanthemum, water hyacinth, monazite, zinc oxide and calcium carbonate.

20. The nebulized inhalation liquid for relieving rhinitis as claimed in claim 19, wherein the celery seed is 300-500 parts by weight, the spring onion seed is 40-80 parts by weight, the watercress is 400-600 parts by weight, the crowndaisy Chrysanthemum is 350-550 parts by weight, the water hyacinth is 150-350 parts by weight, the monazite is 400-600 parts by weight, the zinc oxide is 400-600 parts by weight, the calcium carbonate is 300-400 parts by weight, the glycerol is 30-40 parts by weight, the 1,2-propylene glycol is 20-30 parts by weight, and the structured water is 30-60 parts by weight.
